(19) United States
(12) Patent Application Publication
(10) Pub. No.: US 2006/0122635 A1
(43) Pub. Date: Jun. 8, 2006

(54) Storage system for bioabsorbable fasteners

(76) Inventors: Chad D. Naegeli, Andover, MN (US); David B. Herridge, Mendota Heights, MN (US); Christopher J. Sperry, Plymouth, MN (US); David M. Stoen, Mahtomedi, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/004,516

(22) Filed: Dec. 3, 2004

(51) Int. Cl. A61B 17/10

(52) U.S. Cl. 606/142

(57) ABSTRACT

A storage system for maintaining dimensional tolerances of a plurality of bioabsorbable fasteners from a time of formation to a time of use of the fasteners. The storage system includes a structure defining a cavity. The cavity has a cavity cross-section resembling a corresponding cross-section of the plurality of bioabsorbable fasteners and a cavity length adapted to accept the plurality of the bioabsorbable fasteners in a stacked orientation. The cartridge is fabricated of a material having a heat deflection temperature higher than the temperatures encountered during manufacture, sterilization, shipping or storage. The storage system also includes a locking arrangement operably configured to shield the fasteners from a biasing force outside the cavity until the time of use of the fasteners.

### FIELD OF THE INVENTION

The present invention relates generally to the field of surgical fasteners such as surgical staples and clips. More particularly, the present invention relates to a mechanical method and apparatus for constraining and protecting bioabsorbable fasteners from outside forces during sterilization, transportation and storage such that fastener tolerances are maintained prior to use.

### BACKGROUND OF THE INVENTION

When a wound opening in tissue is created either through an intentional incision or an accidental laceration, biological healing of the wound opening occurs when the opposed living tissue surfaces of the wound opening are in close proximity to each other. If the opening is very large or if its location subjects the wound opening to continual movement, a physician will seek to forcibly hold the sides of the wound opening in close proximity so as to promote the healing process. In the case of skin tissue, for example, healing occurs best when the opposing dermal layers of the skin tissue are held in proximity with each other.

While traditional suturing remains a popular method of effectuating closure of wound openings, the use of staples and staplers as a closure technique has become increasingly popular, especially in surgical settings where the opening is created through a purposeful incision. In these settings, the incision tends to make a clean, straight cut with the opposing sides of the incision having consistent and non-jagged surfaces.

Typically, stapling of a skin opening, for example, is accomplished by manually approximating the opposing sides of the skin opening and then positioning the stapler so that a metal staple will span the opening. The stapler is then manipulated such that the staple is driven into the skin with one leg being driven into each side of the skin and the cross-member of the staple extending across the opening external to the skin surface. Generally, the legs of the metal staple are driven into an anvil causing the metal staple to deform so as to retain the skin tissue in a compressed manner within the staple. This process can be repeated along the length of the wound opening such that the entire incision is held closed during the healing process.

One problem with conventional metal staples is that the metal staples must be removed after the healing process of a wound opening is completed. As the sciences of medical and materials technology have advanced over the course of the past century, new bioabsorbable polymers and copolymers have been developed that provide medical professionals with an alternative to metal staples that must be removed. Fasteners made of bioabsorbable materials, sometimes referred to as bioabsorbable or biodegradable, break down or degrade over time in the body, with the residuals being either absorbed or ultimately expelled by the body's natural processes.

While bioabsorbable polymer fasteners are preferable to metal staples because they do not have to be removed, the lack of an effective deformation property of bioabsorbable polymer materials means that these materials cannot rely on the deformation of the staple to compress and retain tissue in a manner similar to a metal staple. For example, after the initial forming steps the bioabsorbable staples and fasteners retain residual stresses that can lead to dimensional variations between similarly formed components if the residual stresses cause the components to relax in an uncontrolled manner. Consequently, different designs for securing bioabsorbable staples have been developed.

Many bioabsorbable staples utilize a retainer or receiver that performs a self-locking function, either integrated with a piercing portion as shown, for example, in U.S. Pat. No. 4,317,451, or separated to form a two piece staple as shown, for example, in U.S. Pat. Nos. 4,805,617 and 5,902,319. U.S. Pat. No. 4,428,376 describes a bioabsorbable staple with a pivoting arm locking feature that permits the arms of the staple to be inserted in an open position and then locked into a closed position after deployment. Some designs utilize lateral projecting barbs for securing the bioabsorbable staple as shown, for example, in U.S. Pat. Nos. 3,716,058; 3,757,629; 4,014,492; 5,105,252 and 5,584,859. These alternative designs for securing bioabsorbable staples have generally precluded the staples from being stored in a ganged manner similar to metal staples. For example, as shown in U.S. Pat. No. 6,120,526, the need to operate the particular self-locking or retaining function of the bioabsorbable staple can prevent the staples from being loaded into the stapler in a side-by-side ganged fashion like conventional metal staples.

Another design for a bioabsorbable staple utilizes a single shaft skewer approach as shown for example, in U.S. Pat. Nos. 5,292,326; 5,389,102; 5,489,287; and 5,573,541 issued to Green et al. The Green et al. patents attempted to overcome the need for self-locking or retaining functions of a bioabsorbable stapling system by employing a handheld apparatus with jaws to proximate, interdigitate and overlap opposing sides of dermal layer tissue along the length of a skin opening. The apparatus then drives a single spike through the interdigitated and overlapped dermal layers of the opposing skin surfaces to secure both sides of the dermal tissue on the single spike. By using a single spike passing through interdigitated tissue, the Green et al. patents attempted to overcome the need for self-locking or closure mechanisms as required by other prior art bioabsorbable fasteners. This approach also permitted the spikes described by Green to be stored in a ganged or stacked manner, more like conventional metal staples.

U.S. Pat. No. 6,726,705 to Peterson et al describes a tissue fastening system that uses bioabsorbable staples that do not need a self-locking or closure mechanism. Unlike the Green et al patents, the Peterson et al. patent utilizes an applicator apparatus for bioabsorbable fasteners that bilaterally drives at least one portion of the fastener through each side of a wound such that the fastener is positioned below an exterior surface of the wound and a portion of the fastener is positioned generally transverse to a vertical interface of the wound. In one embodiment of this patent, an automated fastener delivery and storage mechanism is described in which the bioabsorbable fasteners are stacked vertically in echelon fashion surrounding a guide member. The ganged stack of staples in this embodiment are biased downwardly from a time of manufacture to a time of use using a spring that in use causes the staples in the ganged arrangement to be advanced.

While the tissue fastening apparatus described in the Peterson et al patent represents a significant improvement over prior art bioabsorbable fasteners, the technique described in the Peterson et al patent for ganging bioabsorbable fasteners uses a constant bias force that may cause the stack of bioabsorbable staples to deform, or stick together when subjected to long periods of time or elevated temperatures prior to use. As such, it would be desirable to provide an improved storage system for bioabsorbable fasteners throughout sterilization, packaging and shipment prior to the time of use.

### SUMMARY OF THE INVENTION

The present invention is a system for storing multiple bioabsorbable fasteners in such a manner as to maintain the dimensional tolerances of the bioabsorbable fasteners from a time of formation to a time of use. The system preferably is implemented as a cartridge that comprises a cavity having a cavity cross-section resembling the bioabsorbable fastener and a cavity length for accepting a plurality of the bioabsorbable fasteners in a stacked orientation. The cartridge is fabricated of a material having a heat deflection temperature higher than the temperatures encountered during shipping or storage of the cartridge loaded with fasteners such that relaxation of the bioabsorbable fasteners induced by elevated temperatures during shipment, packaging and assembly processes such as, for example, certain sterilization processes, is constrained by the cartridge. In one embodiment, a locking arrangement is configured as part of the cartridge to shield the fasteners prior to use from a biasing force that is applied at the time of use to eject the fasteners from the cartridge.

The invention also includes a method for maintaining dimensional tolerances of a bioabsorbable fastener from a time of formation to a time of use. The method comprises loading a cartridge with a plurality of bioabsorbable fasteners in a stacked configuration. The cartridge has an elongated cavity, a first end, a second end, and a cavity cross-section that substantially resembles the bioabsorbable fasteners. The cartridge is fabricated of a material having a heat deflection temperature higher than the temperatures encountered during shipping or storage of the cartridge loaded with the fasteners such that relaxation of the bioabsorbable fasteners induced by elevated temperatures during shipment, packaging and assembly processes such as, for example, certain sterilization processes, is constrained by the cartridge. In one embodiment, the method includes protecting the fasteners from a biasing force that is applied at the time of use to eject the fasteners from the cartridge.

The invention also includes a medical fastening instrument comprising an instrument body including a biasing member, an insertion member, and a cartridge attached to the instrument body. The cartridge has a continuous cavity adapted to receive a plurality of bioabsorbable fasteners to be stored in stacked relation. The cartridge is fabricated of a material having a heat deflection temperature higher than the temperatures encountered during shipping or storage such that relaxation of the bioabsorbable fasteners induced by elevated temperatures during shipment, packaging and assembly processes such as, for example, certain sterilization processes, is constrained by the cartridge. The biasing member causes the insertion member to interact with the cartridge such that the plurality of bioabsorbable fasteners are selectively ejected from the cartridge. In one embodiment, the instrument also includes a lock for isolating the plurality of fasteners once loaded into the cartridge from the biasing member and the insertion member prior to use of the medical fastening instrument and also from outside forces during shipment and storage of the medical fastening instrument.

In another embodiment, the invention includes an instrument configured to maintain the dimensional tolerances of a bioabsorbable fastener. The instrument comprises a cartridge having a cavity for receiving a plurality of fasteners. The cavity is defined by a first surface, a second surface opposite the first surface, at least one rear surface joining the first surface and the second surface, and a forward surface opposite the rear surface, the forward surface including a protrusion at the midpoint of the forward surface. The instrument also includes an insertion head coupled to the cartridge and positioned to align the material to be fastened and a lock for isolating the plurality of fasteners from a biasing force that is used to eject the fasteners at a time of use.

In yet another embodiment, the invention includes a method for maintaining dimensional tolerances of a bioabsorbable fastener. The method includes loading a cartridge with a plurality of bioabsorbable fasteners in a stacked configuration. The cartridge has a cavity for receiving a plurality of fasteners and is defined by a first surface, a second surface opposite the first surface, at least one rear surface joining the first surface and the second surface, and a forward surface opposite the rear surface, the forward surface including a protrusion at the midpoint of the forward surface. The method also includes installing the cartridge within an instrument body. The cartridge and instrument body form an assembled and operable fastening instrument whereby the bioabsorbable fasteners can be selectively discharged. The method also includes protecting the staples from a biasing force used to discharge the fasteners until a time of use with a locking arrangement.

In a preferred embodiment, the invention includes a medical fastening instrument comprising a body assembly including a biasing member, and a cartridge attached to the body assembly. The cartridge has a cavity for receiving a plurality of fasteners. The cavity includes an entrance and an exit and is defined by a first surface, a second surface opposite the first surface, at least one rear surface joining the first surface and the second surface, and a forward surface opposite the rear surface, the forward surface including a protrusion, at the midpoint of the forward surface. An elongated rod is coupled to the biasing member. The rod is configured to provide a force in the direction of the plurality of fasteners such that the plurality of fasteners are selectively ejected from the exit of the cartridge. A removable lock is configured to prevent movement of the rod until a desired time of use.

Unlike existing mechanical tissue fastening systems, the present invention recognizes the need for and advantages of a storage system that constrains bioabsorbable fasteners throughout sterilization, packaging and shipment such that critical tolerances are insured at the time of use.

### BRIEF DESCRIPTION OF THE DRAWINGS

FIG. 1 shows a perspective view of a wound closure instrument incorporating the present invention.

FIG. 2 shows a front view of a wound closure instrument incorporating the present invention.

FIG. 3 shows an enlarged perspective view of a wound closure instrument incorporating the present invention.

FIG. 4 shows a partial cross-sectional view of the cartridge of the present invention.

FIG. 5 shows a top view of the cartridge of the present invention.

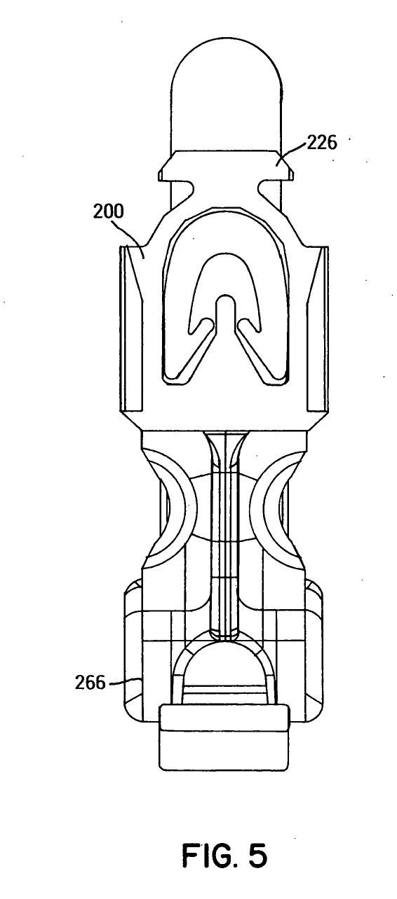

FIG. 6 shows a perspective view of the cartridge of the present invention.

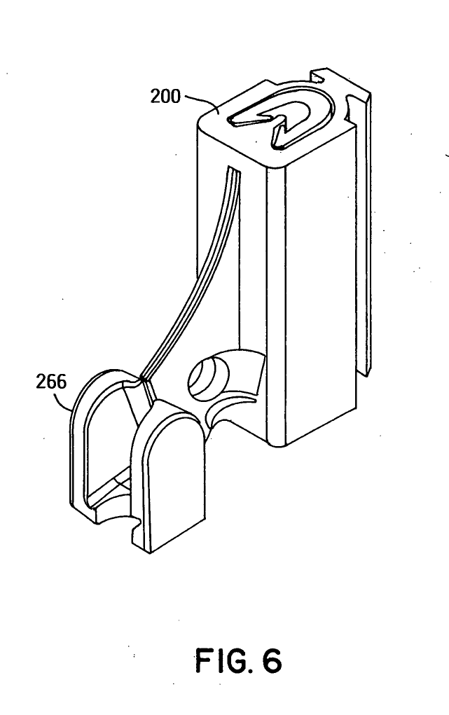

FIG. 7 shows a rear perspective view of the cartridge of the present invention.

FIG. 8 an enlarged perspective view of a wound closure instrument incorporating the present invention.

FIG. 9 is a side view of an instrument incorporating the present invention.

FIG. 10 is a partial front perspective view of an instrument incorporating the present invention.

FIG. 11 is a side partial view of an instrument incorporating the present invention.

FIG. 12 is an enlarged cross-sectional perspective view of an instrument incorporating the present invention.

FIG. 13 is a partial perspective view of a wound closure instrument incorporating the present invention.

FIG. 14 is an exploded view of a wound closure instrument incorporating the present invention.

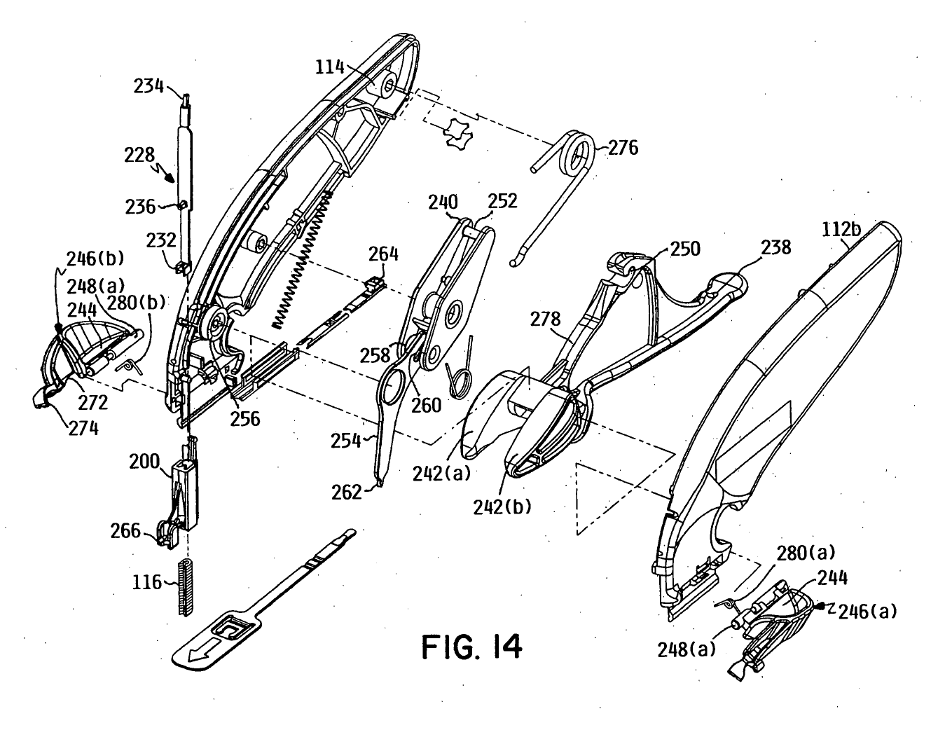

FIG. 15 is a perspective view of the applicator assembly in accordance with the present invention.

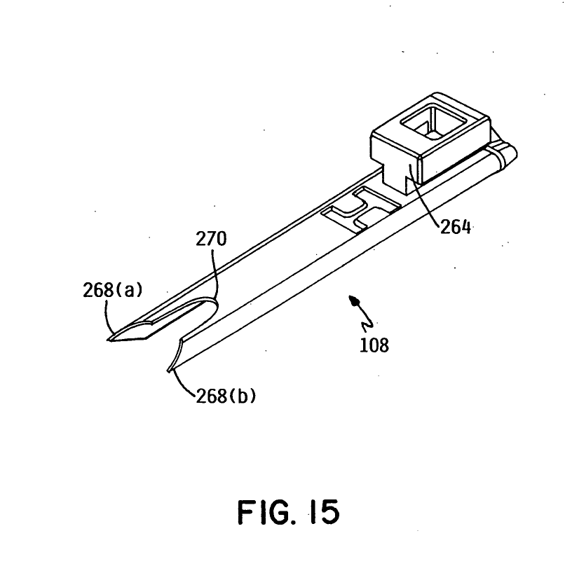

FIG. 16 is a shows a partial cross-sectional view of an alternative embodiment of the cartridge of the present invention.

### DETAILED DESCRIPTION OF THE PREFERRED EMBODIMENTS

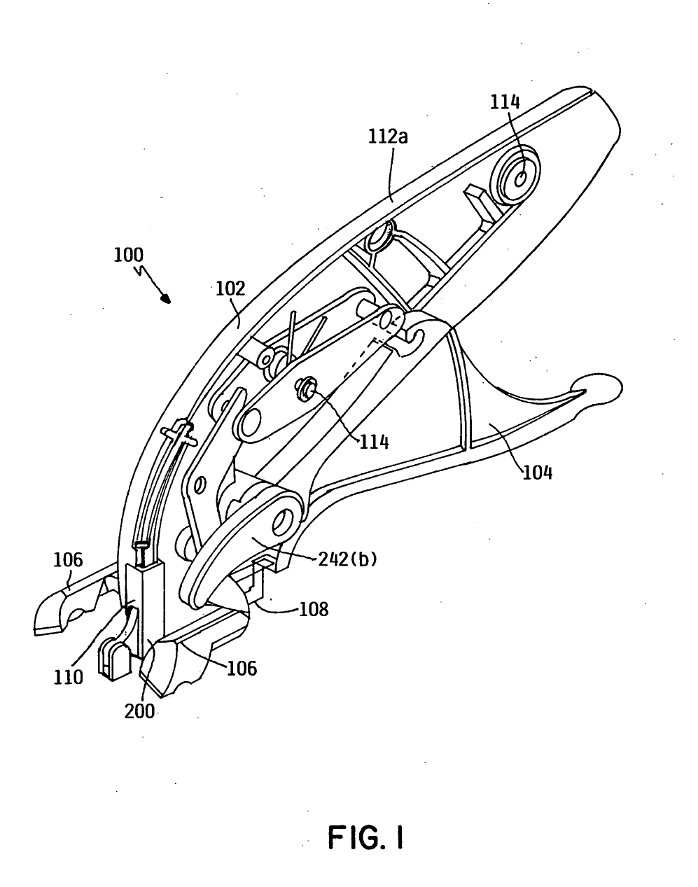

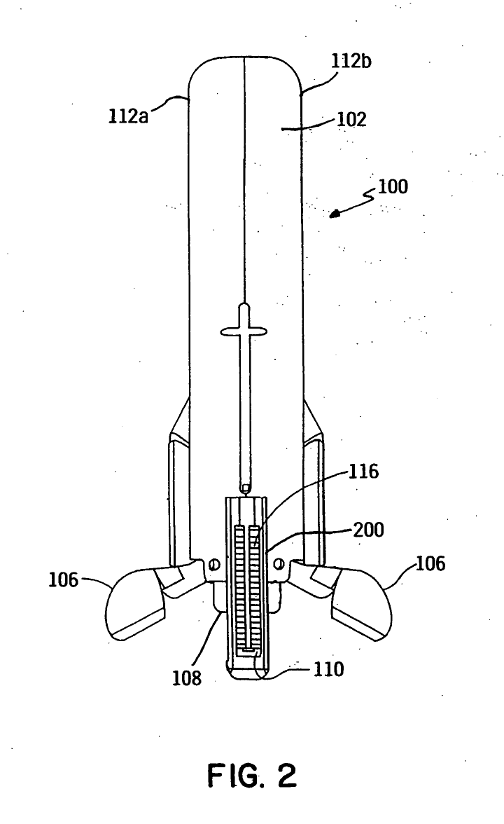

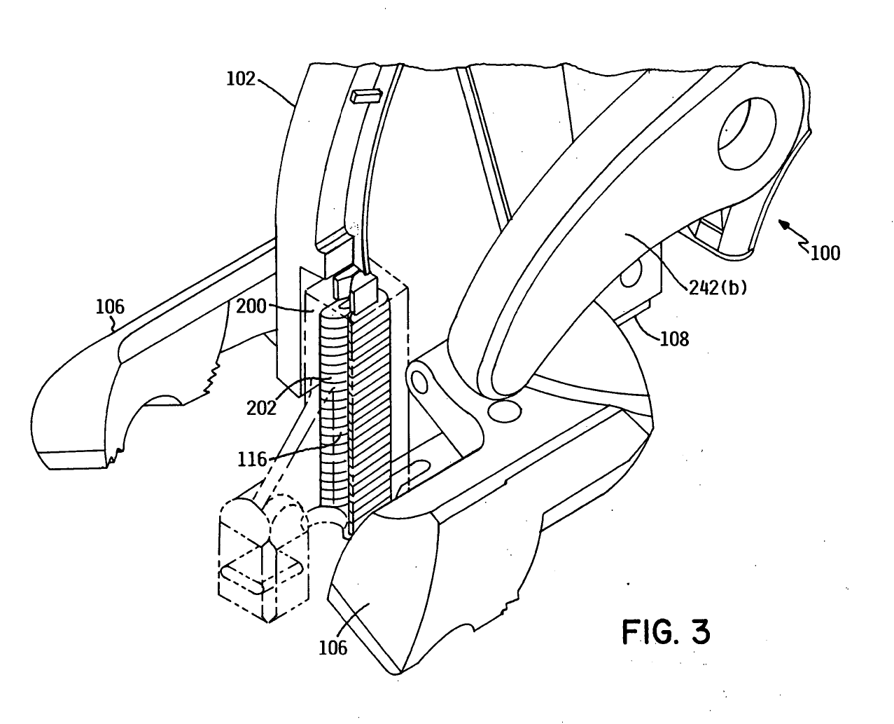

A wound closure instrument 100 is depicted in FIGS. 1, 2, and 3. For example, wound closure instrument 100 can take the form of the Insorb® Subcuticular Stapler as supplied by Incisive Surgical, Inc. of Plymouth, Minn. and as further described in U.S. Pat. No. 6,726,705 and pending U.S. patent application Ser. Nos. 10/448,838, 10/607,497 and 29/202,831, all of which are incorporated by reference in their entirety. Wound closure instrument 100 is comprised of body assembly 102, a trigger assembly 104, a tissue manipulation assembly 106, an applicator assembly 108, and a fastener assembly 110.

Body assembly 102 preferably comprises a clam shell design with a first molded portion 112a and a second molded portion 112b that can be snapped together, thermally bonded, adhesively bonded or connected via a plurality of fastening members 114. Body assembly 102 is preferably fabricated from plastic, although a variety of materials may be used while remaining within the scope of the invention.

Fastener assembly 110 comprises a plurality of bioabsorbable fasteners 116, for example the dynamic bioabsorbable fasteners described in U.S. patent application Ser. No. 10/603,397, which is incorporated by reference in its entirety, and a fastener cartridge 200. Bioabsorbable fasteners 116 are stored within fastener cartridge 200 in a stacked configuration.

Examples of bioabsorbable materials from which bioabsorbable fasteners 116 can be formed include poly(dl-lactide), poly(l-lactide), polyglycolide, poly(dioxanone), poly(glycolide-co-trimethylene carbonate), poly(l-lactide-co-glycolide), poly(dl-lactide-co-glycolide), poly(l-lactide-co-dl-lactide), poly(caprolactone) and poly(glycolide-co-trimethylene carbonate-co-dioxanone). Other polymer, synthetic or biological materials which are designed for initial structural integrity and have the capability of breaking down over time in the body could also be utilized.

It has been discovered that, when used in connection with the fastening apparatus of the preferred embodiment, in addition to having the desired property of breaking down over time in the body, these bioabsorbable materials also have a tendency to deform in response to pressures applied over an extended period of time.

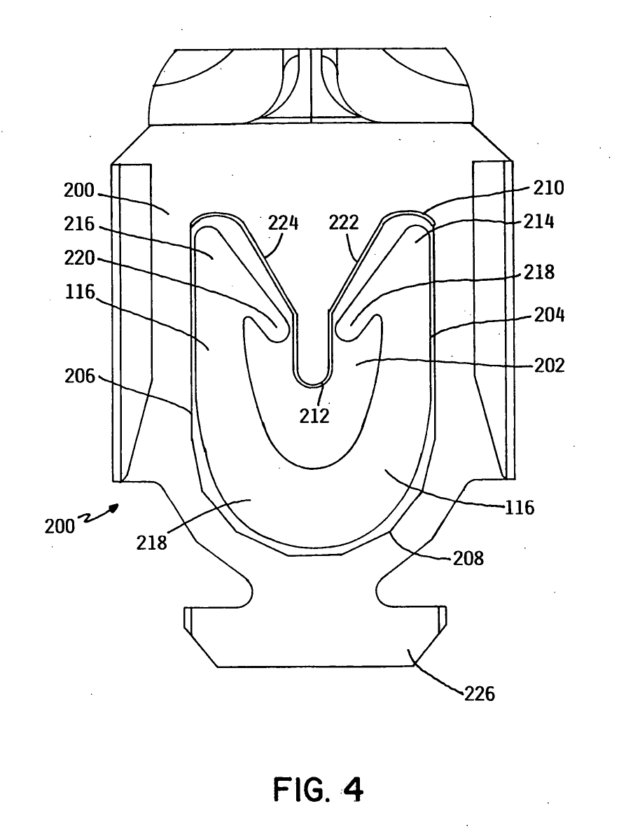

FIG. 4 shows a top cross-sectional view of the fasteners 116 disposed in the cartridge 200. Cartridge 200 includes a cavity 202 that is defined by a first surface 204, a second surface 206, a rear surface 208, and a forward surface 210. Forward surface 210 includes a protrusion 212 located at the midpoint of the forward surface 210. Protrusion 212 is shown extending into cavity 202, toward rear surface 208, between first surface 204 and second surface 206. In one embodiment, forward surface 210 includes kanted portions 222 and 224 extending toward protrusion 212.

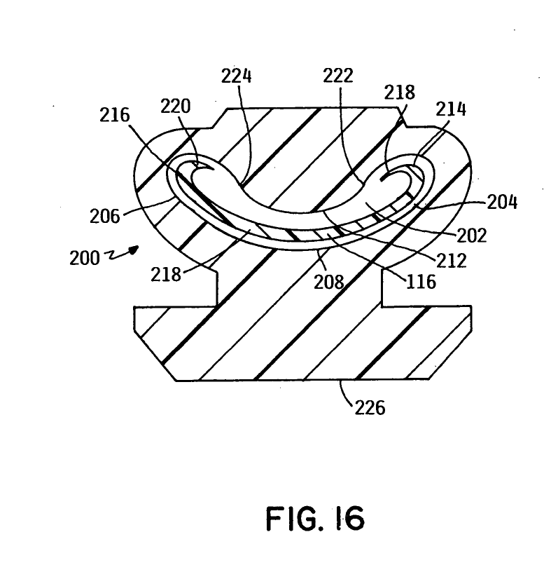

FIG. 16 shows a top cross-sectional view of a dynamic fastener 116 disposed in a cartridge embodiment 200. A description of dynamic fasteners is disclosed in U.S. patent application Ser. No. 10/603,397, which is hereby incorporated by reference in its entirety. Cartridge 200 includes a cavity 202 that is defined by a first surface 204, a second surface 206, a rear surface 208, and a forward surface 210. Forward surface 210 includes a protrusion 212 located at the midpoint of the forward surface 210. Protrusion 212 is shown extending into cavity 202, toward rear surface 208, between first surface 204 and second surface 206. In one embodiment, forward surface 210 includes kanted portions 222 and 224 extending toward protrusion 212.

The dimensions of a preferred embodiment of cavity 202 will now be discussed. Kanted portions 222 and 224 define an angle of between 30 and 90 degrees with respect to one another, preferably between 45 and 75 degrees, more preferably between 55 and 65 degrees. The width of protrusion 212 along the dimension extending into cavity 202 is between 0.010 and 0.042 inches, preferably between 0.018 and 0.034 inches. The width of cavity 202 from first surface 204 to second surface 206 is between 0.130 and 0.150 inches, preferably between 0.138 and 0.142 inches. The maximum height of cavity 202 from kanted portion 222 and rear surface 208 is between 0.190 and 0.210 inches, preferably between 0.197 and 0.203 inches. The distance between the tip of protrusion 212 and rear surface 208 is between 0.090 and 1.110 inches, preferably between 0.092 and 1.108 inches. The rear surface 208 defines a curve with a radius of between 0.060 and 0.080 inches, preferably between 0.065 and 0.075 inches, more preferably 0.070 inches.

Fasteners 116 include a first tip 214, a second tip 216, and a body 218 that joins first tip 214 and second tip 216. In one embodiment, first tip 214 and second tip 216 include barbs 218 and 220, respectively.

In a preferred embodiment, fasteners 116 are constrained within cavity 202 by first surface 204 and second surface 206. This aspect of the invention is desirable for maintaining a constant distance between the first tip 214 and second tip 216. Fasteners 116 may also be constrained by any or all of the rear surface 208, forward surface 210, kanted portions 222, 224 and protrusion 212.

During operation and use, downward pressure is preferably applied to the plurality of fasteners 116 by rod 228 and biasing member 230. Rod 228 includes plug 232 configured to apply pressure to the plurality of fasteners 116 in cavity 202. Biasing member 230 couples with rod 228 at knob 234. The pressure generated by biasing member 230 against the interior of body assembly 102 and rod 228 generates a downward force by plug 232 against the plurality of fasteners 116. It will be recognized that other arrangements for application of a biasing force to the plurality of fasteners 116 could also be utilized whereby the function of the biasing member 230 and rod 228 are combined, such as a spring or a flexible metal member. Alternatively, other structures for applying a biasing force could be used in place of rod 228, such as piston or a crossbar could be utilized.

In a preferred embodiment, rod 228 includes a catch 236. Catch 236 is configured to rest against a corresponding area of molded portion 112 or a removable lock 300. This enables instrument 100 to be shipped fully assembled to facilitate ease of use by a physician. Preferably, catch 236 and the removable lock 300 prevents biasing member 230 from applying force directly to fasteners 116 during shipment or storage so that the fasteners 116 do not deform after prolonged exposure to the spring force. Preferably, instrument 100 is a multi-shot design in which the plurality of fasteners 116 come preloaded in the cartridge 200 with the cartridge 200 assembled as part of the fastener assembly 110, thus eliminating any hand loading of individual fasteners. Alternatively, cartridge 200 may be preloaded or hand loaded and designed for insertion into fastener assembly 110 prior to use.

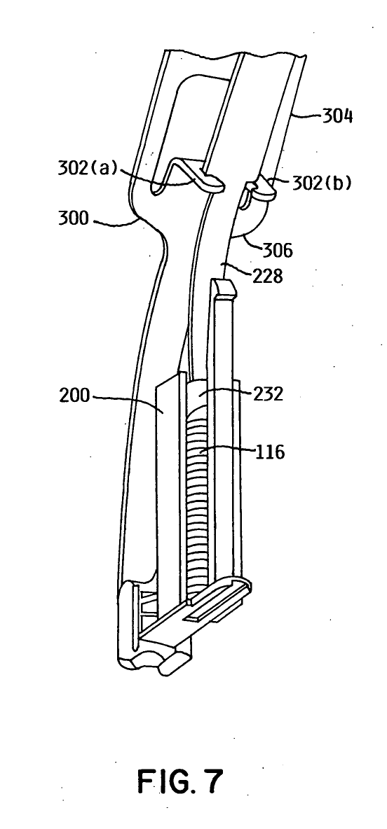

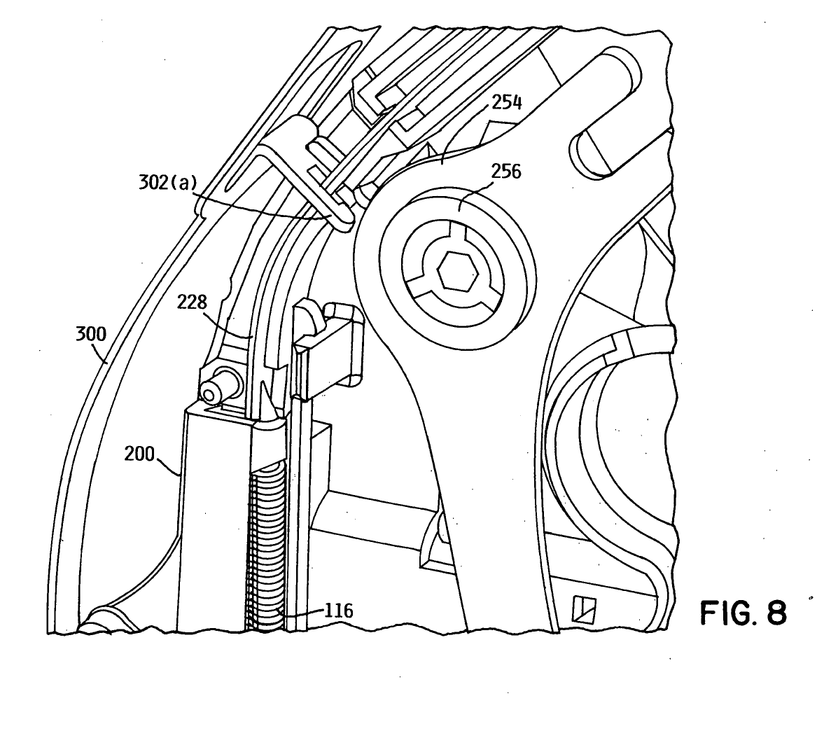

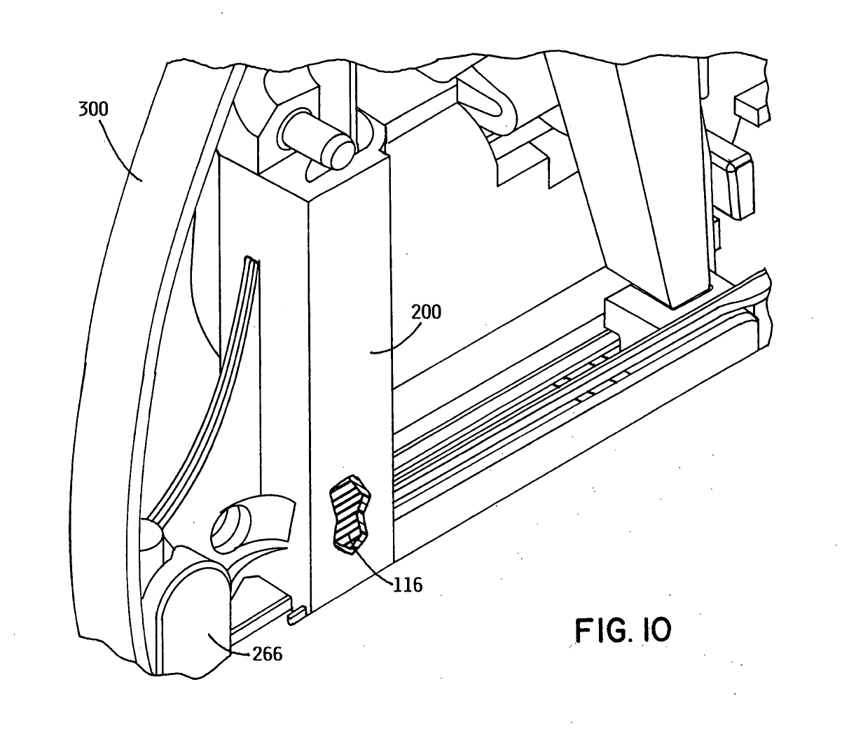

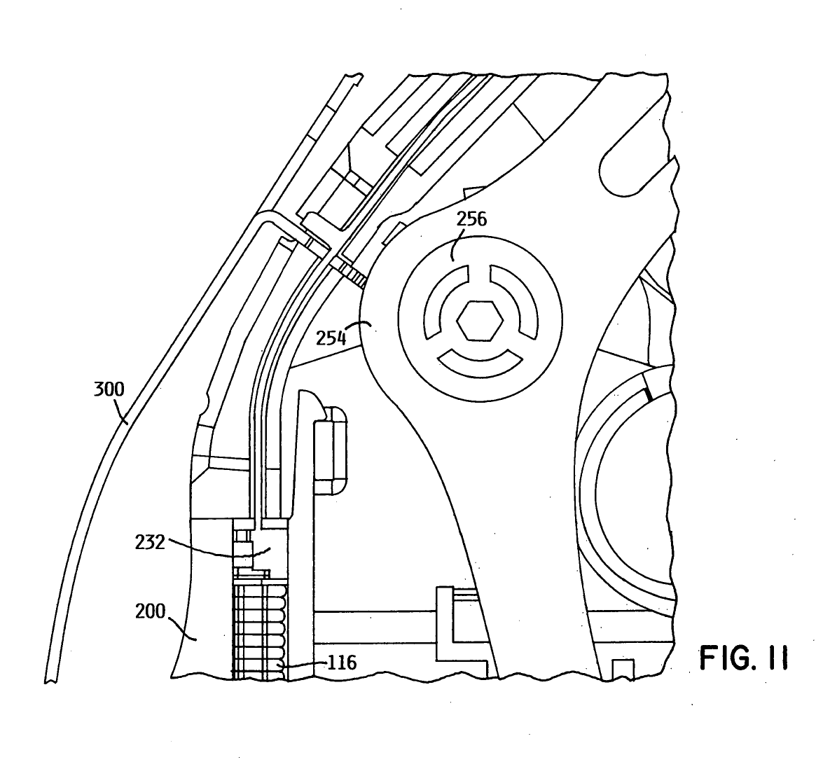

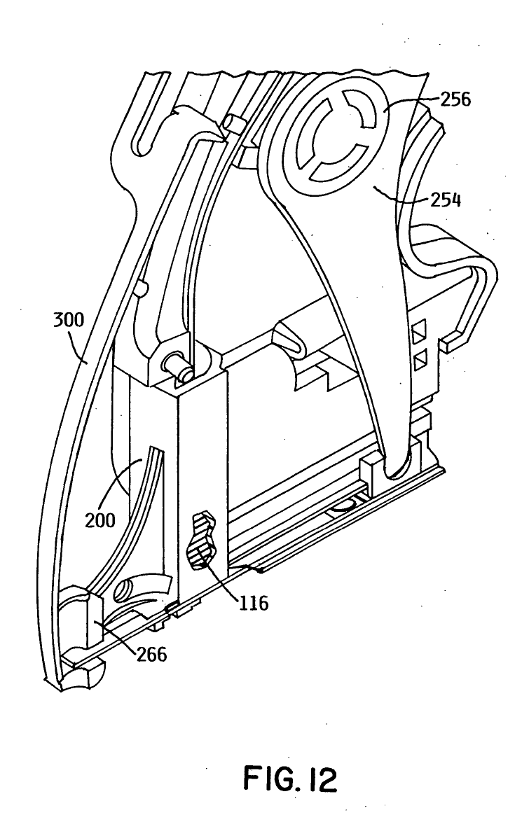

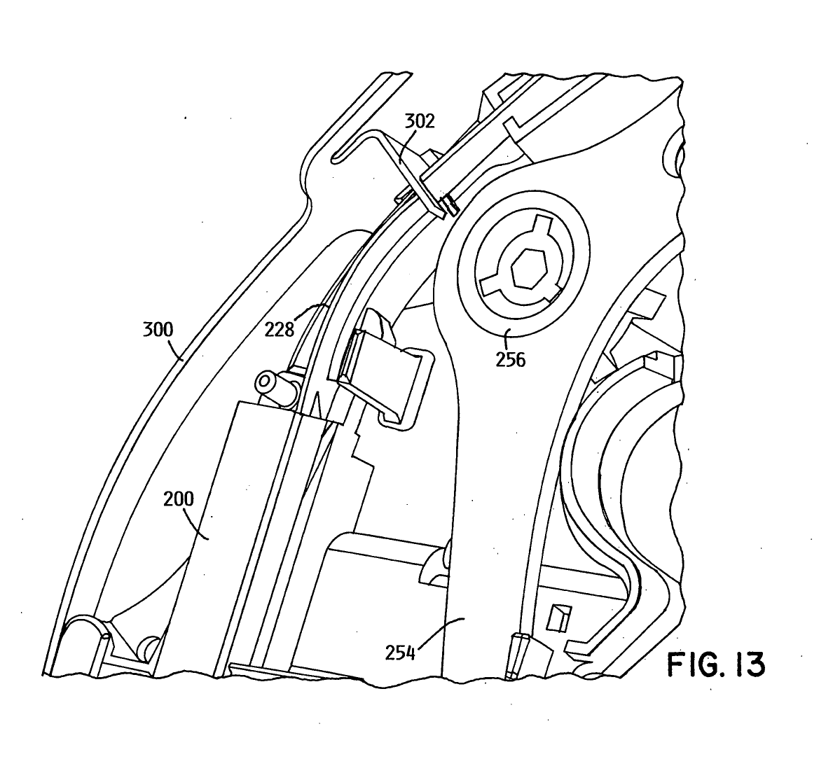

A preferred embodiment of lock 300 will be described in more detail with respect to FIGS. 7-13. Lock 300 includes stopper arms 302(a), 302(b) that extend at approximately a 90 degree angle from lock 300, through an aperture in instrument 100. As shown in FIG. 7, rod 228 is disposed between stopper 302(a) and 302(b). Rod 228 is positioned at an angle approximately perpendicular to stopper 302(a) and 302(b). Rod 228 includes a wide section 304 near the end of the rod 228 adjacent to the biasing member 230. Rod 228 also includes a narrow section 306 between the wide section 304 and the plug 232. As shown in FIG. 7, the stopper 302 of lock 300 prevents the wide section 304 of rod 228 from passing through stopper 302(a) and 302(b). In a preferred embodiment, plug 232 is disposed at least partially in the cavity of cartridge 200 when the movement of rod 228 is secured by lock 300. This mechanism, by its nature, prevents the plurality of fasteners from escaping from the top entrance of the cavity.

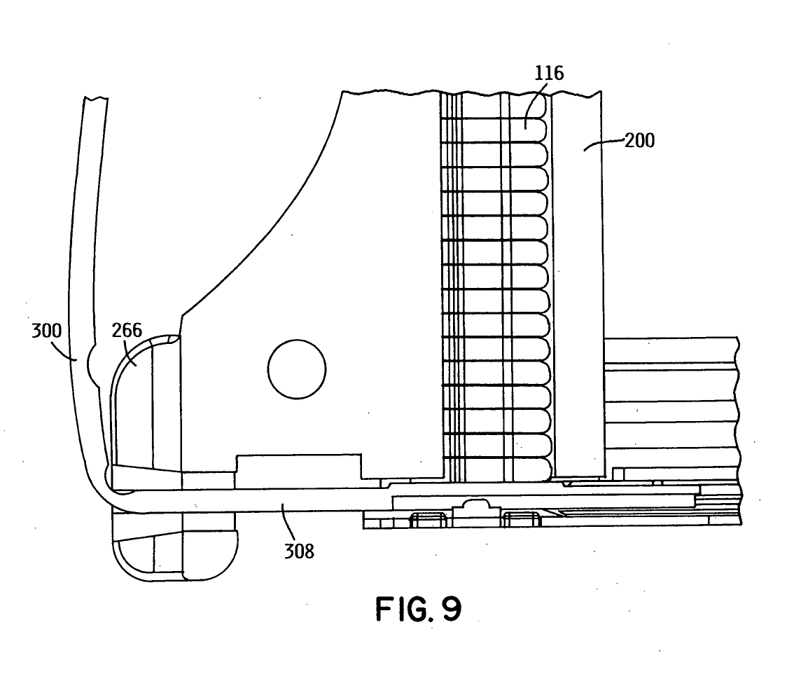

As shown in FIG. 9, lock 300 includes lower section 308 that extends into and through insertion head 266 and below cartridge 200. This feature of the invention holds the plurality of fasteners within the cartridge 200.

When instrument 100 is ready to be used, lock 300 is simply pulled away from instrument 100 such that lower section 308 slides out of insertion head 266. Stopper 302(a), 302(b) move away from rod 228 and enable rod 228 to slide downward due to the pressure of biasing member 230. The pressure of biasing member 230 enables plug 232 to apply pressure to the plurality of staples 116 so that the lowest staple is positioned against the applicator assembly 108. In this configuration, instrument 100 is ready for operation. Preferably, the biasing member 230 is a spring member. Alternatively, arrangements of elastic bands or belts, metal flat springs, or even a gas or liquid pressure activated mechanism could be used to provide the desired biasing force.

Although the present invention has been described with respect to the various embodiments, it will be understood that numerous insubstantial changes in configuration, arrangement or appearance of the elements of the present invention can be made without departing from the intended scope of the present invention. Accordingly, it is intended that the scope of the present invention be determined by the claims as set forth.

1. A storage system for maintaining dimensional tolerances of a plurality of bioabsorbable fasteners from a time of formation of the fasteners to a time of use of the fasteners, the storage system comprising:
structure defining a cavity having a cavity cross-section resembling a corresponding cross-section of the plurality of bioabsorbable fasteners and a cavity length adapted to accept the plurality of the bioabsorbable fasteners in a stacked orientation, the cartridge being fabricated of a material having a heat deflection temperature higher than the temperatures encountered during shipping, or storage; and
a locking arrangement operably configured to shield the fasteners from a biasing force outside the cavity.

2. The storage system of claim 1, further comprising a plurality of bioabsorbable fasteners disposed within the cavity, each of the plurality of bioabsorbable fasteners having a first tip, a second tip, and a body joining the first tip and the second tip.

3. The storage system of claim 2, wherein the plurality of fasteners are fabricated from bioabsorbable polymers and copolymers selected from the group including: poly(dl-lactide), poly(l-lactide), polyglycolide, poly(dioxanone), poly(glycolide-co-trimethylene carbonate), poly(l-lactide-co-glycolide), poly(dl-lactide-co-glycolide), poly(l-lactide-co-dl-lactide), poly(caprolactone) and poly(glycolide-co-trimethylene carbonate-co-dioxanone).

4. A method for storing of a plurality of bioabsorbable fasteners from a time of formation of the fasteners to a time of use of the fasteners comprising:
loading a cartridge with a plurality of bioabsorbable fasteners in a stacked configuration after the time of formation;
protecting the fasteners from a biasing force outside the cavity prior to the time of use; and
at the time of use, permitting the biasing force to aid in selectively positioning sequential ones of the plurality of the bioabsorbable fasteners for a fastening operation.

5. A medical fastening instrument comprising:
an instrument body including a biasing member configured to apply a force,
an insertion member,
a structure operably associated with the instrument body and defining a cavity having a cavity length between opposed ends adapted to accept the plurality of bioabsorbable fasteners in a stacked orientation, one end of the cavity being operably aligned with the insertion member and another end of cavity being operably arranged with the biasing member such that the bioabsorbable fasteners from the cavity are selectively ejected from the instrument one fastener at a time, and
a locking arrangement operably configured to shield the plurality of bioabsorbable fasteners from a force generated by the biasing member from a time that the plurality of bioabsorbable fasteners are loaded into the cavity until prior to a time of use of the medical fastening instrument.

6. The medical fastening instrument of claim 5, further comprising a plurality of bioabsorbable fasteners disposed within the cavity, each of the plurality of bioabsorbable fasteners having a first tip, a second tip, and a body joining the first tip and the second tip.

7. The medical fastening instrument of claim 6, wherein the plurality of fasteners are fabricated from bioabsorbable polymers and copolymers selected from the group including: poly(dl-lactide), poly(l-lactide), polyglycolide, poly(dioxanone), poly(glycolide-co-trimethylene carbonate), poly(l-lactide-co-glycolide), poly(dl-lactide-co-glycolide), poly(l-lactide-co-dl-lactide), poly(caprolactone) and poly(glycolide-co-trimethylene carbonate-co-dioxanone).

8. A method of maintaining a shape of a bioabsorbable fastener during sterilization, the method comprising:
loading a cartridge with a plurality of bioabsorbable fasteners, the cartridge having structure defining a cavity with a cross-section resembling a corresponding cross-section of the plurality of bioabsorbable fasteners and a cavity length adapted to accept the plurality of the bioabsorbable fasteners in a stacked orientation, the cartridge being fabricated of a material having a heat deflection temperature higher than the heat deflection temperature of the plurality of bioabsorbable fasteners; and
sterilizing the cartridge loaded with the plurality of bioabsorbable fasteners by subjecting the cartridge to an elevated temperature that is less than the heat deflection temperature of the plurality of bioabsorbable fasteners.

9. The method of claim 8, further comprising:
installing the cartridge in a body of an applicator that is adapted to deliver the plurality of bioabsorbable staples one at a time for a fastening operation.

10. A method for maintaining dimensional tolerances of a plurality of bioabsorbable fasteners from a time of formation of the fasteners to a time of use of the fasteners, the method comprising:
loading a cartridge with a plurality of bioabsorbable fasteners in a stacked configuration in a cavity defined in the cartridge for receiving the plurality of bioabsorbable fasteners, the cavity including an entrance and an exit, the cavity defined by:
a first surface,
a second surface opposite the first surface,
at least one rear surface joining the first surface and the second surface, and
a protrusion opposite the rear surface; and
operably installing the cartridge within an instrument body, such that the cartridge and instrument body form an assembled and operable fastening instrument whereby the bioabsorbable fasteners can be selectively discharged and such that the bioabsorbable fasteners are effectively protected from outside forces that affect dimensional tolerances up to a time of deployment of the fasteners.

11. The method of claim 10, wherein the rear surface closely resembles an outer profile of a body of the plurality of fasteners.

12. The method of claim 11, wherein the first surface and the second surface are generally flat.

13. The method of claim 12, wherein a first tip of the plurality of bioabsorbable fasteners is disposed between the first surface and the protrusion.

14. The method of claim 13, wherein a second tip of the plurality of bioabsorbable fasteners is disposed between the second surface and the protrusion.

15. A medical fastening instrument comprising:
a body assembly including a biasing member,
a cartridge operably assembled with the body assembly, the cartridge having a cavity for receiving a stacked arrangement of plurality of fasteners, the cavity defined by:
an entrance,
an exit,
a first surface,
a second surface opposite the first surface,
at least one rear surface joining the first surface and the second surface, and
a forward surface opposite the rear surface, the forward surface including a protrusion, at generally a midpoint of the forward surface,
an elongated member operably coupled to the biasing member, the elongate member configured to provide a force though the entrance of the cartridge onto the plurality of fasteners such that the plurality of fasteners are selectively ejected from the exit of the cartridge, and
a removable lock configured to prevent movement of the elongated member until a time of use of the instrument, thereby preventing application of the force onto the plurality of fasteners.

16. The instrument of claim 15, wherein the lock includes a lower section for preventing any one of the plurality of fasteners from passing through the exit of the cavity.

17. The instrument of claim 16, wherein the lock includes a stopper that prevents any one of the plurality of fasteners from passing through the entrance of the cavity.

18. The instrument of claim 17, wherein the elongate member is a rod that includes a plug on the side of the rod opposite the biasing member.

19. The instrument of claim 18, wherein the plug is at least partially disposed in the entrance of the cavity.

20. The instrument of claim 19, wherein the rod includes a wide section and a narrow section, wherein the stopper engages the rod at the narrow section.
